(19) United States
(12) Patent Application Publication
(10) Pub. No.: US 2016/0206459 A1
(43) Pub. Date: Jul. 21, 2016

(54) DEVICES FOR GASTROINTESTINAL BYPASS HAVING TISSUE INGROWTH FEATURES

(71) Applicant: ValenTx, Inc., Maple Grove, MN (US)

(72) Inventors: Johann Neisz, Minneapolis, MN (US); James Wright, Santa Barbara, CA (US)

(21) Appl. No.: 15/000,959

(22) Filed: Jan. 19, 2016

Related U.S. Application Data
(62) Division of application No. 14/139,859, filed on Dec. 23, 2013, now abandoned.
(60) Provisional application No. 61/745,580, filed on Dec. 22, 2012.

(51) Int. Cl. A61F 5/00; A61F 2/00; A61F 2/04

(52) U.S. Cl. CPC A61F 5/0076; A61F 2/04; A61F 2/0077; A61F 2002/044; A61F 2002/045; A61F 2002/009

(57) ABSTRACT

Gastrointestinal bypass devices are described. In one embodiment, a gastrointestinal bypass device includes a liner having a proximal portion configured to be at least partially positioned in the esophagus and a distal portion configured to be at least partially positioned in the intestine. The gastrointestinal bypass device also includes a framework coupled to the proximal portion of the liner and a tissue ingrowth area formed at the proximal portion of the liner.

### CROSS-REFERENCE TO RELATED APPLICATIONS

This application is a divisional of U.S. patent application Ser. No. 14/139,859, filed Dec. 23, 2013, which claims the benefit of U.S. provisional patent application No. 61/745,580, filed Dec. 22, 2012. These applications are hereby incorporated by reference in their entireties.

### BACKGROUND

Diabetes, heart disease, and other obesity-related conditions may be treated surgically with bariatric procedures such as jejuno-ileal bypass, jejuno-colic bypass, biliopancreatic diversion, gastric bypass, and gastroplasty. These procedures may be effective for weight control and treatment of chronic conditions. However, these procedures carry with them substantial shortcomings, including the risk of infection and other risks accompanying surgery. Some of these procedures induce radical permanent changes to the gastrointestinal anatomy, thus foreclosing subsequent surgical intervention.

What is needed are devices and methods that use non-surgical techniques that avoid the risks associated with gastrointestinal bypass surgery.

### SUMMARY

Gastrointestinal bypass devices are described. In one embodiment, a gastrointestinal bypass device includes a liner having a proximal portion configured to be at least partially positioned in the esophagus and a distal portion configured to be at least partially positioned in the intestine. The gastrointestinal bypass device also includes a framework coupled to the proximal portion of the liner and a tissue ingrowth area formed at the proximal portion of the liner.

### BRIEF DESCRIPTION OF THE DRAWINGS

FIGS. 1A-1D show various embodiments of a tissue ingrowth device 4100.

FIG. 1E shows one embodiment of a framework 4170.

FIG. 1F-1I shows additional various embodiments of a tissue ingrowth device 4100.

### DESCRIPTION

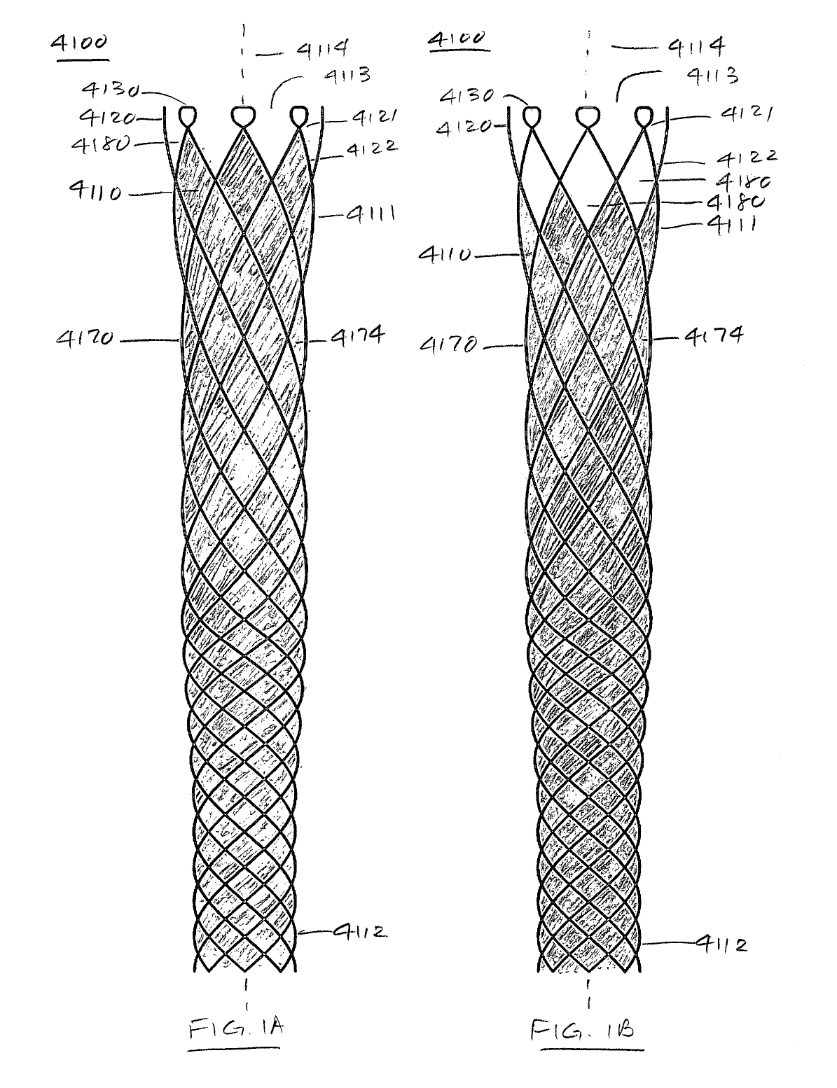

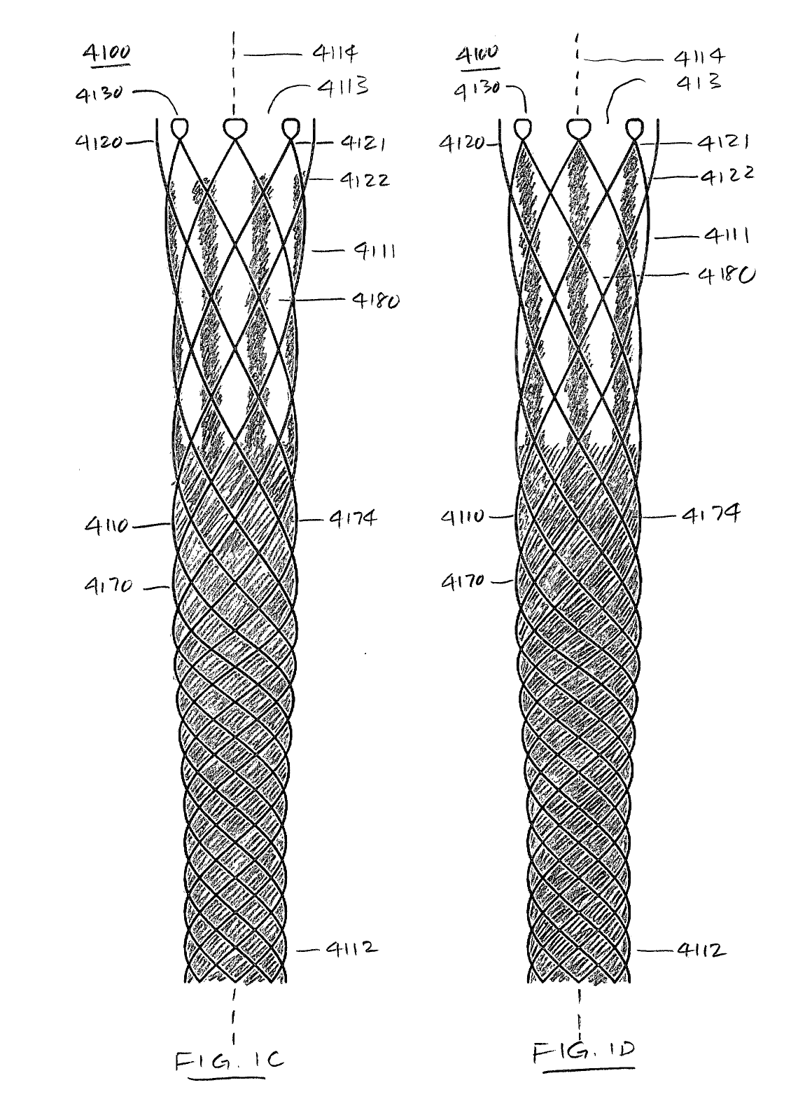

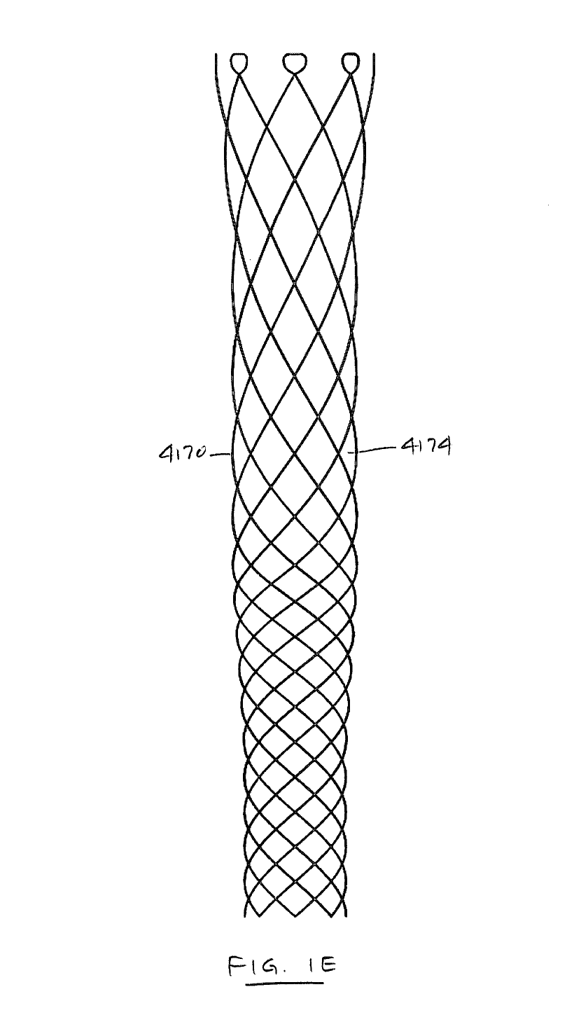

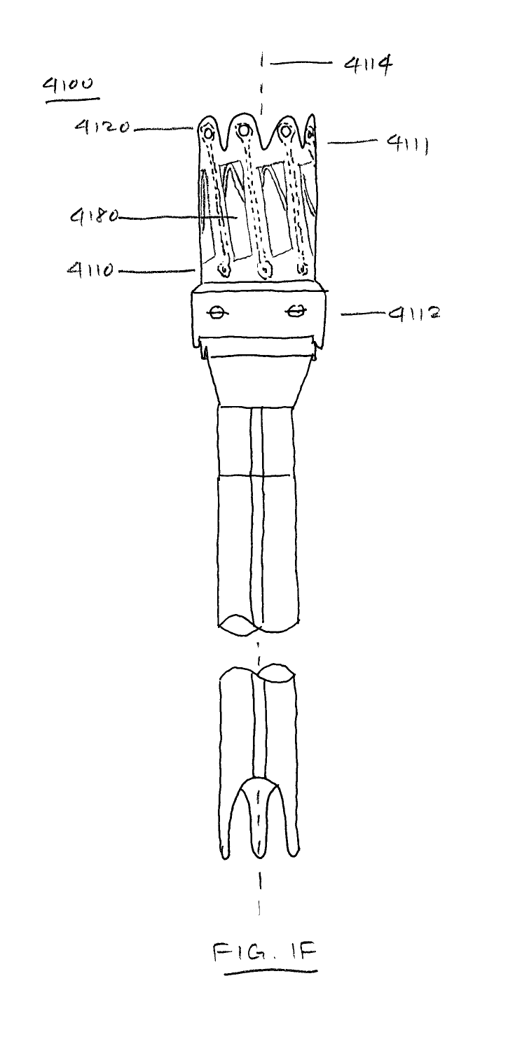

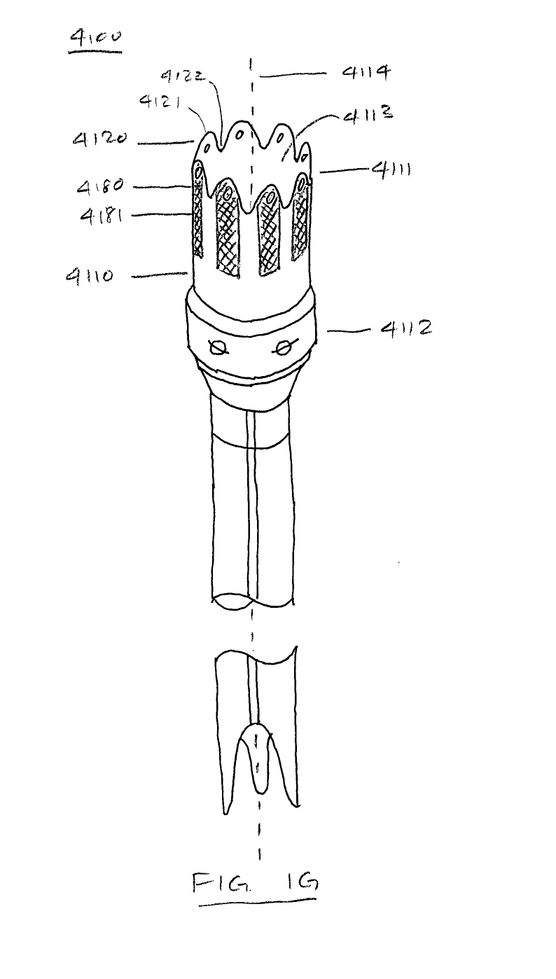

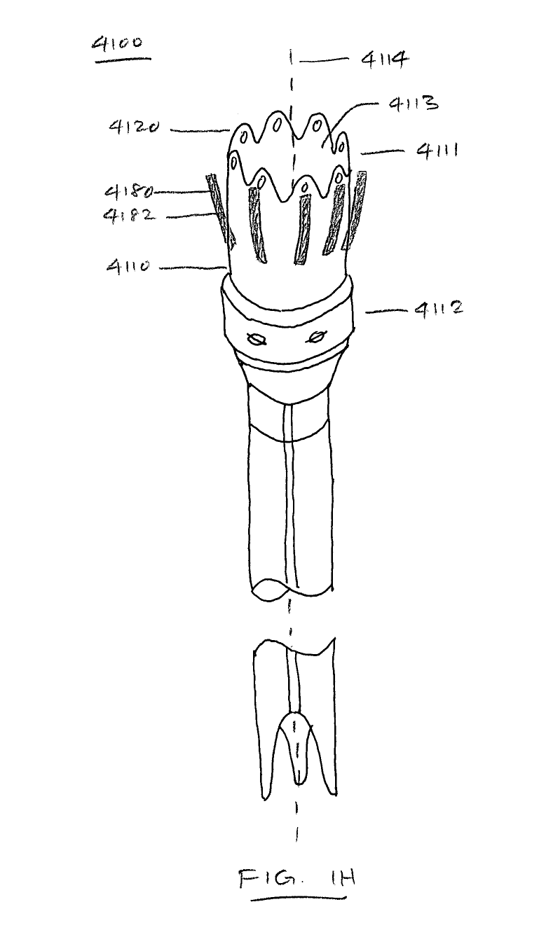

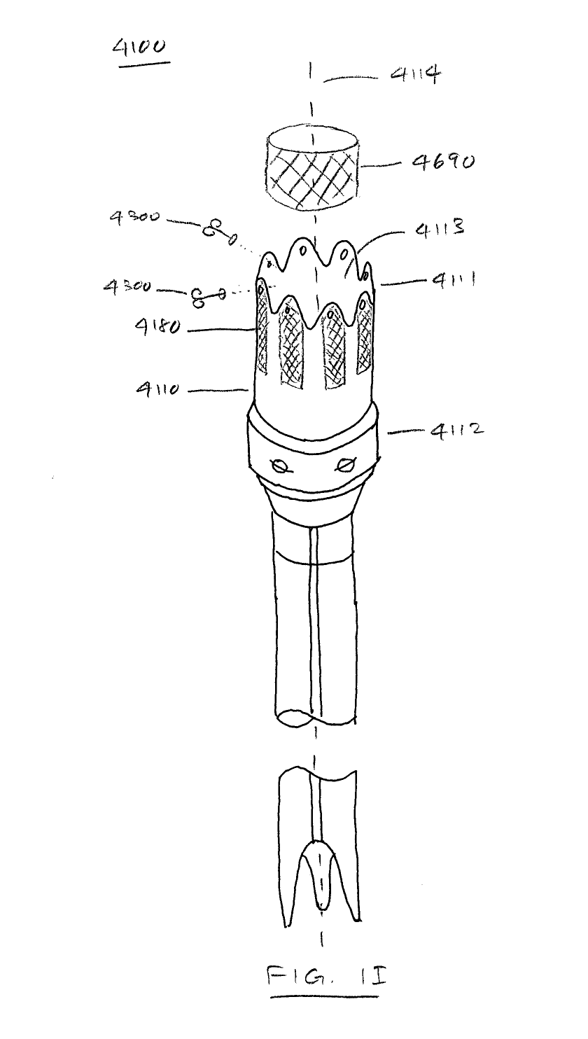

FIGS. 1A-1D show various embodiments of a tissue ingrowth device 4100.

Tissue ingrowth device 4100 may be any device configured to be attached at least partially within a bodily lumen. For example, tissue ingrowth device 4100 may be a gastrointestinal bypass device or gastrointestinal cuff similar to those described in U.S. patent application Ser. No. 13/896,838, which is hereby incorporated by reference in its entirety. When used as a gastrointestinal bypass device, tissue ingrowth device 4100 may have a length of 80 cm to 600 cm. When used as a gastrointestinal cuff, tissue ingrowth device 4100 may have a length of approximately 20 mm to 80 mm.

As another example, tissue ingrowth device 4100 may be a GERD device or other device configured to be attached at least partially within the esophagus, stomach, and/or intestine. Tissue ingrowth device 4100 may be generally cylindrical, conical, or any other suitable configuration or shape.

Tissue ingrowth device 4100 may include a liner 4110, a proximal edge 4120, a framework 4170, and one or more tissue ingrowth areas 4180.

Liner 4110 may include a proximal portion 4111, a distal portion 4112, a lumen 4113, and a longitudinal axis 4114. Liner 4110 may be tubular and may have a uniform width. Alternatively, liner 4110 may taper or change in width. Liner 4110 may be made of material that is flexible. This flexibility allows the attachment area, such as the lower esophageal sphincter, to open and close substantially normally. Liner 4110 may be made of a material that is thin, allowing it to collapse into a smaller profile. This smaller profile allows the attachment area, such as the lower esophageal sphincter, to close substantially normally, and also helps liner 4110 to be collapsed for delivery. Liner 4110 may include a single layer of material, or any number of layers. Liner 4110 may be at least semi-permeable to liquids and/or solids. This semi-permeability may allow some food to reach the stomach, and may help the stomach maintain some residual functionality. This semi-permeability may allow medications to reach the stomach. Liner 4110 may be made of DACRON or any other suitable material.

Proximal edge 4120 may be zig-zag, straight, angled, wavy, scalloped, or any other configuration. A zig-zag proximal edge 4120 may include a plurality of peaks 4121 and valleys 4122 formed at the edge of proximal portion 4111 of liner 4110. Peaks 4121 and valleys 4122 may be of uniform shape and size. Alternatively, peaks 4121 and valleys 4122 may be of varying shapes and sizes. A zig-zag proximal edge 4120 allows peaks 4121 to open wider than the rest of liner 4110. This enhances conformance of proximal edge 4120 to an inside of the bodily lumen, such as the esophagus, and reduces the amount of food and liquids which pass outside of tissue ingrowth device 4100 and into the stomach. Reducing the amount of food or liquid that passes outside of tissue ingrowth device 4100 can in some cases advantageously improve clinical endpoints (e.g., improved weight loss, improved glycemic, lipid profile, blood pressure, etc.). Valleys 4122 may include a webbing which may be thinner than liner 4110. A zig-zag proximal edge 4120 reduces bunching of liner 4110 when the esophagus is closed, and reduces the profile of liner 4110 when the esophagus is closed.

Optionally, a coupling mechanism, e.g., anchor holes 4130 may be formed at or near proximal portion 4111 of liner 4110. Anchor holes 4130 are configured to receive tissue anchors. Anchor holes 4130 may be marked with a contrasting color, radiopaque marker, or other means to aid visualization. Anchor holes 4130 may be marked with stitching, ink, or other suitable marking. Anchor holes 4130 may be used as a placement template for tissue anchors. Anchor holes 4130 may be evenly spaced about a circumference of liner 4110. Evenly spaced anchor holes 4130 may help to distribute forces among the tissue anchors, prevent concentration of forces in a small number of tissue anchors, and enhance conformance of liner 4110 to an inside (e.g., luminal wall) of the esophagus. Alternatively, anchor holes 4130 may be spaced in any manner about a circumference of liner 4110. Anchor holes 4130 may be substantially coplanar. Anchor holes 4130 may be arranged in a plane substantially perpendicular to longitudinal axis 4114, or angled to longitudinal axis 4114. A substantially coplanar arrangement may help to prevent concentration of forces in a small number of tissue anchors. Alternatively, anchor holes 4130 may be arranged in a staggered fashion.

Anchor holes 4130 may be formed as portions of framework 4170 elsewhere within or near peaks 4121 or proximal edge 4120, and/or formed in liner 4110. As an example, for a zig-zag proximal edge 4120 with peaks 4121 and valleys 4122, anchor holes 4130 may be formed by portions of framework 4170 extending proximal to peaks 4121, as shown in FIGS. 1A-1C.

FIG. 1E shows one embodiment of a framework 4170. Framework 4170 may be coupled to liner 4110 between layers of liner. Alternatively, framework 4170 may be coupled to an inner surface or an outer surface of liner 4110. Framework 4170 may be coupled to liner 4110 with sutures, adhesives, or other suitable ways. Framework 4170 may include a stent or wire mesh. Framework 4170 may have a weave of uniform or variable density. For example, framework 4170 may have a weave that is less dense and less resistant to collapsing where it coincides with the lower esophageal sphincter, such as at or near proximal portion 4111 of liner 4110. Framework 4170 may have a weave that is more dense and more resistant to collapsing elsewhere. Framework 4170 includes spaces 4174. Alternatively, framework 4170 may have any desired wall pattern. Alternatively, framework 4170 may include a ring or other suitable device.

Framework 4170 provides an outward bias to enhance conformance of liner 4110 to the tissue wall of the bodily lumen, such as the esophagus. This outward bias may be large enough to open liner 4110 when the esophagus opens, but not so large as to prevent the esophagus from closing, such as where framework 4170 coincides with the lower esophageal sphincter. Framework 4170 is not necessarily meant to hold tissue ingrowth device 4100 in the bodily lumen, such as the esophagus. Framework 4170 may have a geometry, such as length and thickness, selected to create a desired amount of outward bias. Framework 4170 may be made of a material selected to create a desired amount of outward bias. Framework 4170 may be made of a material selected to elicit a foreign body response. Framework 4170 may be made of plastic such as PEEK, metal, or any other suitable material.

Framework 4170 may provide a structure on which liner 4110 is formed. For example, framework 4170 may be coated with a biocompatible layer to form liner 4110. For example, framework 4170 may be dipped or covered in polyurethane, silicone, or other suitable material to form a flexible, elastic coating which serves as liner 4110.

Framework 4170 may include one or more portions that form at least part of peaks 4121 and/or valleys 4122. Framework 4170 may include one or more portions that form at least part of anchor holes 4130. As an example, for a zig-zag proximal edge 4120 with peaks 4121 and valleys 4122, framework 4170 may include loops of any suitable size and configuration which extend proximal to peaks 4121, as shown in FIGS. 1A-1C, and serve as anchor holes 4130.

One or more tissue ingrowth areas 4180 may be formed or coupled at or near proximal portion 4111 of liner 4110. Tissue ingrowth areas 4180 may be configured to allow and/or promote tissue from the luminal wall to grow at least partially through and/or attach to tissue ingrowth device 4100. Tissue ingrowth areas 4180 may include one or more portions of framework 4170 that are not covered by liner 4110. For example, a tissue ingrowth area 4180 may include a portion of framework 4170 exposed by cutting away a circumferential ring of liner 4110 at or near proximal portion 4111 of liner 4110, as shown in FIG. 1A. As another example, tissue ingrowth areas 4180 may include circumferentially alternating portions of framework 4170 exposed by cutting away portions of liner 4110 at or near one or more peaks 4121, as shown in FIGS. 1B-1C, or valleys 4122, as shown in FIG. 1D. These circumferentially alternating tissue ingrowth areas 4180 may be substantially diamond-shaped, rectangular, elongate, round, or any other suitable shape. These circumferentially alternating tissue ingrowth areas 4180 may be configured to allow the bodily lumen to open and close substantially normally by promoting ingrowth at some points for attachment to the tissue wall, but discouraging ingrowth at other points, thus allowing the liner 4110 to bunch up and collapse. For example, for a zig-zag proximal edge 4120 with peaks 4121 and valleys 4122, tissue ingrowth areas 4180 may be positioned at peaks 4121, as shown in FIGS. 1B-1C, to allow tissue from the tissue wall to grow into at least a portion of peaks 4121. Alternatively, for a zig-zag proximal edge 4120 with peaks 4121 and valleys 4122, tissue ingrowth areas 4180 may be positioned at valleys 4122, as shown in FIG. 1D.

FIG. 1F shows another embodiment of a tissue ingrowth device 4100. Tissue ingrowth device 4100 may include tissue ingrowth areas 4180 formed by cutting away or removing one or more portions of the liner between the struts of a gastrointestinal bypass device or gastrointestinal cuff similar to those described in U.S. patent application Ser. No. 13/896,838.

FIG. 1G shows another embodiment of a tissue ingrowth device 4100. Tissue ingrowth device 4100 may have a scalloped edge 4120. Tissue ingrowth device 4100 may include tissue ingrowth areas 4180 comprised of one or more patches 4181 coupled to an exterior surface of liner 4110. Patches 4181 may be coupled at or near proximal portion 4111 of liner 4110, or any other suitable location, of devices as described above in FIGS. 1A-1D, or a gastrointestinal bypass device or gastrointestinal cuff similar to those described in U.S. patent application Ser. No. 13/896,838. Patches 4181 may be coupled at or near peaks 4121 of scalloped edge 4120. Alternatively, patches 4181 may be coupled at or near valleys 4122 of scalloped edge 4120.

Patches 4181 may be substantially diamond-shaped, rectangular, elongate, round, or any other suitable shape. Patches 4181 may be arranged in a circumferentially alternating, circumferential, checkerboard, or other suitable configuration. Patches 4181 may be made of a material selected to elicit a foreign body response. Patches 4181 may be made of plastic such as PEEK, metal, or any other suitable material. Patches 4181 may include a wire mesh or weave. Patches 4181 may coupled to liner 4110 with suture, glue, or any other suitable way. Patches 4181 may be coupled to liner 4110 along a portion or all of a perimeter of patches 4181. Liner 4110 underneath patches 4181 may be intact to limit or prevent tissue ingrowth from occluding lumen 4113.

FIG. 1H shows another embodiment of a tissue ingrowth device 4100. Tissue ingrowth device 4100 may include tissue ingrowth areas 4180 comprised of one or more flaps or extensions 4182 coupled to or extending from any point along liner 4110 of devices as those described above in FIGS. 1A-1D, or a gastrointestinal bypass device or gastrointestinal cuff similar to those described in U.S. patent application Ser. No. 13/896,838. Flaps or extensions 4182 may include structures and/or materials or otherwise be configured to allow tissue ingrowth.

Tissue ingrowth areas 4180 may include spaces 4174 of a size selected to promote tissue ingrowth. For example, tissue ingrowth areas 4180 may include spaces 4174 of a size ranging from about 6 μm to 2.5 mm. Tissue ingrowth areas 4180 may also include spaces 4174 of a size large enough to permit macrophages to pass through. For example, tissue ingrowth areas 4180 may include spaces 4174 of a size greater than about 100 μm.

FIG. 1I shows another embodiment of tissue ingrowth device 4100. Tissue ingrowth device 4100 may include temporary fixation devices and/or non-temporary fixation devices.

One or more temporary fixation devices may be used to hold liner 4110 in place for a sufficient period of time to allow tissue from the tissue wall to grow at least partially through and/or attach to tissue ingrowth areas 4180. Temporary fixation devices may include a self-expanding stent 4690 placed inside lumen 4113 of liner 4110 in the vicinity of tissue ingrowth areas 4180. Stent 4690 may be configured to exert sufficient outward force to hold tissue ingrowth areas 4180 against the tissue wall. Stent 4690 may be removed later, or may be made of a biodegradable material. Stent 4690 may be attached to liner 4110 with sutures or other suitable device which can be cut away or removed when stent 4690 is removed. Temporary fixation devices may include sutures used to couple tissue ingrowth device 4100 to the tissue wall. Such sutures may be removed later, or may be made of a biodegradable material. Temporary fixation devices may include one or more tissue anchors 4300 used to couple tissue ingrowth device 4100 to the tissue wall. Tissue anchors 4300 may be cut away and/or removed after sufficient tissue ingrowth has occurred, or may be made of a biodegradable material. Tissue anchors 4300 as temporary fixation devices may be coupled to a coupling mechanism such as anchor holes 4130. Tissue anchors 4300 may be similar to those described in U.S. Pat. No. 8,070,743, and U.S. patent application Ser. Nos. 12/137,473 and 13/896,838, which are hereby incorporated by reference in their entirety, or any other suitable tissue anchor.

One or more non-temporary fixation devices may be used instead of, or in conjunction with, temporary fixation devices. Non-temporary fixation devices may include sutures used to couple tissue ingrowth device 4100 to the tissue wall. Such sutures may be left in place, even after sufficient tissue ingrowth has occurred. Non-temporary fixation devices may include one or more tissue anchors 4300 used to couple tissue ingrowth device 4100 to the tissue wall. Tissue anchors 4300 may be left in place, even after sufficient tissue ingrowth has occurred. Tissue anchors 4300 as non-temporary fixation devices may be coupled to a coupling mechanism such as anchor holes 4130.

Non-temporary fixation devices may provide fixation of tissue ingrowth device 4100 to the tissue wall in addition the fixation provided by tissue ingrowth at tissue ingrowth areas 4180. As an example, tissue anchors 4300 may be used when tissue ingrowth device 4100 is placed in a bodily lumen. In the beginning, tissue may grow into tissue ingrowth areas 4180 fairly rapidly, over the course of as little as a few days, while tissue anchors 4300 may take more time to heal and/or be encapsulated by the tissue wall. During an initial period of time, more fixation of tissue ingrowth device 4100 may be provided by tissue ingrowth areas 4180 than by tissue anchors 4300. Over time, more fixation of tissue ingrowth device 4100 may be provided by tissue anchors 4300 than by tissue ingrowth areas 4180.

While the foregoing has been with reference to particular embodiments of the invention, it will be appreciated by those skilled in the art that changes in these embodiments may be made without departing from the principles and spirit of the invention, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or subcombinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described above. For all of the embodiments described above, the steps of any methods need not be performed sequentially.

1. A tissue ingrowth device configured to be coupled within a bodily lumen having a tissue wall, the tissue ingrowth device comprising:
a framework;
a liner covering the framework, the liner having a zig-zag edge formed at a proximal portion of the liner, the zig-zag edge including a plurality of peaks and valleys; and
a plurality of tissue ingrowth areas formed at or near one or more of the peaks of the liner, the tissue ingrowth areas circumferentially alternating with covered areas, wherein the tissue ingrowth areas include portions of the framework that are not covered by the lining and allow tissue ingrowth from the tissue wall, and wherein the covered areas are covered by the lining and discourage tissue ingrowth from the tissue wall.

2. The device of claim 1, wherein the framework includes spaces.

3. The device of claim 2, wherein the spaces have a size of approximately 6 μm to 2.5 mm.

4. The device of claim 1, wherein the framework is made of a material which allows tissue ingrowth.

5. The device of claim 1, wherein the framework has a weave of variable density.

6. The device of claim 1, wherein the lining includes a coating.

7. The device of claim 1, further comprising a temporary fixation device.

8. The device of claim 7, wherein the temporary fixation device includes a self-expanding stent configured to be placed inside a lumen of the liner and configured to exert sufficient outward force to hold the tissue ingrowth areas against the tissue wall.

9. The device of claim 1, further comprising a non-temporary fixation device.

10. The device of claim 9, wherein the non-temporary fixation device includes a tissue anchor configured to couple the framework and/or the liner to the tissue wall.

11. A tissue ingrowth device configured to be coupled within a bodily lumen having a tissue wall, the tissue ingrowth device comprising:
a framework;
a liner coupled to the framework; and
one or more tissue ingrowth patches coupled to an exterior surface of the liner, the tissue ingrowth patches arranged in a circumferentially alternating configuration.

12. The device of claim 11, wherein the tissue ingrowth patches are made of a material which includes spaces.

13. The device of claim 12, wherein the spaces have a size of approximately 6 μm to 2.5 mm.

14. The device of claim 11, wherein the tissue ingrowth patches are made of a material which allows tissue ingrowth.

15-16. (canceled)

17. The device of claim 11, further comprising a temporary fixation device.

18. (canceled)

19. The device of claim 11, further comprising a non-temporary fixation device.

20. The device of claim 19, wherein the non-temporary fixation device includes a tissue anchor configured to couple the framework and/or the liner to the tissue wall.

21-24. (canceled)

25. A gastrointestinal bypass device, comprising:
a liner having a proximal portion configured to be at least partially positioned in the esophagus and a distal portion configured to be at least partially positioned in the intestine;
a framework coupled to the proximal portion of the liner; and
a tissue ingrowth area formed at the proximal portion of the liner.

26. The gastrointestinal bypass device of claim 25, wherein the tissue ingrowth area includes one or more portions of the framework that are not covered by the liner.

27. The gastrointestinal bypass device of claim 25, wherein the tissue ingrowth area includes a circumferential portion of the framework that is not covered by the liner.
